(19) United States
(12) Patent Application Publication
(10) Pub. No.: US 2022/0246287 A1
(43) Pub. Date: Aug. 4, 2022

(54) SYSTEM AND METHOD FOR DYNAMICALLY MANAGING SURGICAL PREFERENCES

(71) Applicant: DIGNITY HEALTH, San Francisco, CA (US)

(72) Inventors: Brian DAWSON, Phoenix, AZ (US); Connie BLACKFORD, Phoenix, AZ (US); Gregory ANSON, Phoenix, AZ (US)

(21) Appl. No.: 17/621,912

(22) PCT Filed: Jun. 22, 2020
(86) PCT No.: PCT/US2020/038961

§ 371 Date: Dec. 22, 2021

Related U.S. Application Data
(60) Provisional application No. 62/865,870, filed on Jun. 24, 2019.

(51) Int. Cl. G16H 40/20; G16H 10/60

(52) U.S. Cl. CPC G16H 40/20; G16H 10/60

(57) ABSTRACT

A Surgical Preference Management System is used to collect and store surgical preferences (“preference cards”) specifying resources desired to perform medical tasks. Information concerning costs and surgical outcomes are continuously monitored and associated with preference cards for those tasks. Analyses of cost, outcome, and other related data are used to identify opportunities to improve outcomes, costs, and other metrics by applying suggested modifications to preference cards. Selected analysis results and suggestions are presented to users within a user interface which allows users to adopt suggestions and make other modifications which cause preference cards to be immediately updated and supplied to other connected medical systems.

### CROSS-REFERENCE TO RELATED APPLICATIONS

This application claims priority to and incorporates by reference U.S. Provisional Application No. 62/865,870 entitled “DYNAMICALLY MANAGING SURGICAL PREFERENCES” and filed on Jun. 24, 2019.

### BACKGROUND OF THE INVENTION

Surgical procedures require the use of human and other physical resources, including physicians, nurses, and surgical technicians; use of particular facilities (e.g., operating rooms, surgical suites, procedure rooms, etc.); equipment; and supplies which may be either reusable or disposable. The particular resources required vary depending upon the procedure. The resources required for a given procedure may also vary due to many other factors. In many cases, one set of factors is the preferences of a particular surgeon. For example, different surgeons may use different surgical approaches for the same procedure. In addition, surgeons may have preferences for certain surgical implements and supplies, including the number, type, and manufacturer of those items. Conventionally, surgeons may communicate their preferences for a particular procedure so the required facilities, staff, implements, supplies, etc. are prepared in advance. Frequently, surgeons or other staff will prepare “preference cards,” which specify a surgeon's preferences for a particular procedure or class of procedures. These preference cards are then used to procure and arrange the surgeon's desired tools, equipment, and supplies for the procedure. Increasingly, preference cards, or equivalent means of communicating surgeon preferences, may be generated, stored, and centrally managed within computing environments.

### BRIEF SUMMARY

In an embodiment, a method of managing surgical preferences comprises receiving preference cards via processing circuitry of a surgical preference management system (SPMS). Each preference card includes a set of surgical preferences associated with a particular procedure type and a particular surgeon. The surgical preferences specifying quantities of surgical resources desired by the surgeon when performing the particular procedure type.

The method further comprises determining a cost for each surgical resource in each preference card by identifying a procurement record corresponding to that surgical resource in an enterprise resource planning system (ERPS) based on a resource description of that surgical resource in that preference card, and retrieving an estimated cost from the identified record.

The method further comprises retrieving a record indicating that a particular surgical resource may be substituted by an alternative surgical resource from substitute preference data stored by the SPMS; and displaying a recommendation via a user interface supplied by the SPMS to alter a preference card selected by a user of the SPMS.

The recommendation is based on one of the following: (1) determining, using a record retrieved from the ERPS, that a cost of the alternative surgical resource is lower than a cost of the particular surgical resource; (2) determining, using a record retrieved from an outcomes database stored by the SPMS, that a procedure outcome metric of the alternative surgical resource is better than an procedure outcome metric of the particular surgical resource; (3) determining, using utilization data collected by the SPMS and associated with the selected preference card, that average costs of one or more resources consumed during surgical procedures associated with the selected preference card is greater than numbers of the one or more specified in the selected preference card by more than a first predetermined threshold; and (4) determining, using utilization data collected by the SPMS and associated with the one or more other preference cards, that average costs of surgical procedures associated with the selected preference card are greater than average costs of surgical procedures associated with the one or more other preference cards.

The method further comprises receiving an instruction from the user via the user interface to implement one or more of the recommended alterations to the particular preference card and implementing the one or more recommended alterations by updating the selected preference card; and causing the updated selected preference card to be used to automatically procure surgical resources for a surgical procedure associated with the selected preference card.

The above features and advantages of the present invention will be better understood from the following detailed description taken in conjunction with the accompanying drawings.

### BRIEF DESCRIPTION OF THE DRAWINGS

The drawings described herein constitute part of this specification and include exemplary embodiments of the present invention which may be embodied in various forms. It is to be understood that in some instances, various aspects of the invention may be shown exaggerated or enlarged to facilitate an understanding of the invention. Therefore, drawings may not be to scale.

FIG. 1 depicts an example environment in which embodiments disclosed herein may be used.

FIGS. 2A and 2B depict example user interface features of certain embodiments.

FIG. 3 depicts example user interface features for accessing a physician profile in certain embodiments.

FIG. 4A depicts example user interface features for viewing surgical preference cards in certain embodiments.

FIG. 4B depicts a block-level flow diagram of a procedure performed in certain embodiments to produce user interface features shown in FIG. 4A.

FIG. 5 depicts example user interface features for viewing surgical preference card information in certain embodiments.

FIG. 6 depicts example user interface features for viewing metrics related to surgical procedures performed by a physician in certain embodiments.

FIG. 7A depicts example user interface features for viewing results of analyzing a physician's surgical history and reviewing recommendations based on that history.

FIG. 7B depicts a block-level flow diagram of a procedure performed in certain embodiments to produce user interface features shown in FIG. 7B.

### DETAILED DESCRIPTION

The described features, advantages, and characteristics may be combined in any suitable manner in one or more embodiments. One skilled in the relevant art will recognize that the circuit may be practiced without one or more of the specific features or advantages of a particular embodiment. In other instances, additional features and advantages may be recognized in certain embodiments that may not be present in all embodiments.

Reference throughout this Specification to “one embodiment,” “an embodiment,” or similar language means that a particular feature, structure, or characteristic described in connection with the embodiment is included in at least one embodiment. Thus, appearances of the phrase “in one embodiment,” “in an embodiment,” and similar language throughout this specification may, but do not necessarily, all refer to the same embodiment.

References throughout this Specification to “physician,” “surgeon,” and similar terms are meant to refer to medical practitioners and other individuals (or groups) who communicate surgical preferences affecting the performance of medical procedures. For example, the individual may be a doctor, a nurse, or a nurse practitioner. Similarly, references to a “surgery” or an “operation” may refer to any related medical procedure. References to an “operating room” or any similar term may refer to any room or area where a medical procedure may be performed. References to “users” may refer to administrative or other personnel as well as medical practitioners. For example, in some cases a user may be an administrative professional reviewing information pertaining to preferences and performance of medical practitioners. Such a user may input information on behalf of a medical practitioner or review information for administrative purposes. References to the medical field are intended for purposes of illustration. It should be understood that the principles disclosed herein are broadly applicable to other fields where individuals express resource allocation preferences (e.g., supply purchasing preferences) for various tasks. Thus, nothing herein is intended to limit the claimed improvements to the medical field.

Conventional systems and methods for managing surgical preferences have disadvantages. For example, variation in surgical preferences can make management of surgical supplies and equipment difficult. In some instances, the choice of supplies, (which may include disposable items as well as items such as surgical implants) and implements for a particular procedure or type of procedure, may be significantly correlated with patient outcomes and/or the cost of performing the procedure. Standardization of procedures across physicians and hospitals is often highly desirable, both from the perspective of patient health, as well as from the perspective of reducing costs to hospitals, patients, and insurers.

Accordingly, the present disclosure addresses these and other shortcomings by gathering and storing preference cards electronically and coordinating with existing medical information systems (i.e., electronic health records, scheduling systems, billing systems, etc.) to monitor and gather data related to those preference cards on an ongoing basis to identify opportunities to modify preference cards to achieve improved surgical outcomes, immediate costs savings by reducing waste, and indirect cost savings due to increased automation. Relevant data and suggestions are presented directly to users who directly drive costs and outcomes in an integrated environment which allows changes to be implemented immediately.

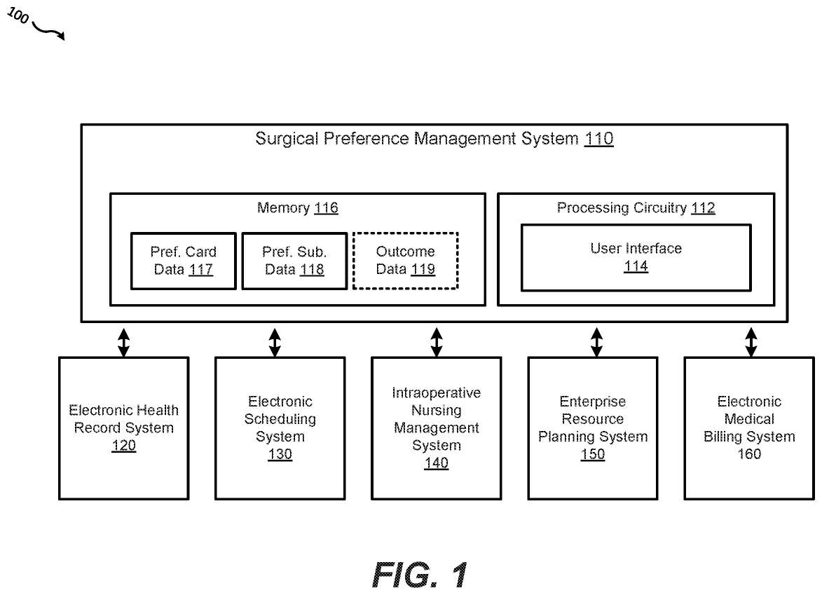

FIG. 1 shows an example environment 100 in which embodiments may be practiced. A Surgical Preference Management System (SPMS 110) implemented by a computer server includes processing circuitry 112 and (not shown) data communication capabilities enabling SPMS 110 to communicate data with remote computer systems. The processing circuitry 112 provides a user interface 114. The SPMS 110 also includes memory 116 storing various data (described further below), including the preference card data 117, the preference substitution data 118, and (optionally, in some embodiments) the outcome data 119. The SPMS 110 may exchange information with other systems, such as an Electronic Health Record System (EHRS 120), an Electronic Scheduling System (ESS 130), an Intraoperative Nursing Management System (INMS 140), an Enterprise Resource Planning System (ERPS 150), and an Electronic Medical Billing System (EMBS 160). These systems may be distinct computing systems communicating over a local network or the Internet. In some instances, a single computing device or distributed computing (“cloud”) system may host more than one of the systems above and similar systems. Users interact with the SPMS 110 via the user interface 114 to perform various actions described herein, such as inputting surgical preference data to create preference cards, viewing preference cards, and reviewing other information, such as the results of analyses and suggestions based on data collected by the SPMS 110 associated with various preference cards or users. The user interface may be provided in cooperation with one or more computing environments. For example, a user may interact with the SPMS 110 through a software application, such as a web browser or other application running on a remote user device using Microsoft Windows, MacOS, Linux, or the like. The user may also interact with the SPMS 110 via a mobile device application, such as an iOS or Android application running on a mobile phone or tablet that is configured to communicate with SPMS 110 via a suitable communications network or interface.

Initially, one or more users enter preference data representing preference cards into the SPMS 110 and, specifically, memory 116 of SPMS 110. Typically, the preference cards include a physician identifier and a procedure or procedure type indicating a particular preference card. The procedure may be identified by its common procedural technology (CPT) code and/or related international classification of disease (ICD) code(s) (e.g., ICD-10 classification codes). The CPT and ICD codes are codes (e.g., alpha-numeric) that can be used to identify particular diseases or procedures. When stored in memory 116 (and, specifically, preference card data 117 repository), the preference card may be stored in a database entry that includes entries containing specific identifiers (e.g., CPR or ICD codes) that can identify diseases or procedures associated with the preference card. The preference card further includes identifiers and/or descriptions of particular surgical implements and supplies that a practitioner may wish to have on-hand when performing the procedure as well as identifications of quantities required for the various surgical implements and supplies. The preference card may include information specifying specific facilities needed or specific pieces of equipment (for example, an aspirator, respirator, imaging equipment, etc.). In some cases, the preference data may include personnel preferences as well (e.g., two surgical technicians, a nurse, and an anesthesiologist).

In some embodiments, the system may receive information from the EHRS 120 or ESS 130 indicating that a procedure has been scheduled. Using information retrieved from these or other systems, the system may retrieve an appropriate preference card and relay information from that preference card to various individuals or systems involved in managing the procedure, as discussed further below. If no preference card for the given combination of procedure and physician(s) exists, the SPMS 110 may prompt a user to create a new preference card. The SPMS 110 may auto-populate a suggested preference card using information from similar preference cards associated with similar procedures and/or similar physicians. In some embodiments, the SPMS 110 may present the user with multiple suggested preference card templates, along with an indication of the relative costs of implementing each of the suggested preference cards. In some embodiments, the SPMS 110 presents the user with a suitable preference card having a relatively low (or absolute lowest) cost for the procedure. In some embodiments, a surgeon may search based on keywords or medical billing and/or diagnosis codes and inspect matching preference cards presented via the user interface 114 to manually identify an appropriate template preference card.

In some embodiments, once a preference card has been assigned to a procedure, the SPMS 110 may communicate with the INMS 140 to aid in (or automate) generation of an appropriate intraoperative nursing note. For example, the SPMS 110 may be accessed via the user interface 114 using an electronic device in an operating room. The SPMS 110 may prompt a user such as a nurse or surgical technician to indicate to the SPMS 110 whether any modifications have been made to the resources used during a procedure compared to the preference card for that procedure. The SPMS 110 may be configured to accept such changes immediately and update the preference card data 117 and/or to prompt the user via a computing device used by that user (or by other means such as electronic mail or text message) to provide updates at a later time, if updates were not provided during the procedure. Similarly, the SPMS 110 may relay information about a procedure to the ERPS 150 or other system to aid in or automate procurement of required supplies.

The SPMS 110 may also be used to perform various audit functions. For instance, the SPMS 110 can detect that a preference card does not contain sufficient identifying information for various resources to enable billing or other goals and retrieve the necessary detailed information from the INMS 140 after a procedure has been performed using that preference card. For example, a preference code may identify an associated procedure that requires assistance from a separate billing entity (e.g., a procedure may indicate that an anesthesia capability is required for the procedure, but may not indicate the entity that will ultimately provide the anesthesia service or provide information on how that entity is to be billed). The SPMS 110 may also prompt a user to supply missing information. As discussed further below, the SPMS 110 may also receive information from other systems such as the ERPS 150 and/or the EMBS 160 to determine whether the preference card data 117 is accurate and/or sufficiently detailed and retrieve the necessary information if not. For example, for each preference card type (e.g., preference cards for each potential procedure), the SPMS 110 may store a listing of a minimum allowable set of data elements to constitute a completed preference card. When a new preference card is created for a particular procedure type (or an existing card is modified), the SPMS 110 may be configured to compare the data associated with the preference card to the predetermined minimum set of data elements. If the data in the preference is missing any of the minimum set of data elements, the user may be alerted to the missing elements via a notification encoded into user interface 114 and the user can supply the missing elements via user interface 114. Conversely, the SPMS may supply information accessed from other systems and supply the missing information. If, for example, the EMBS 160 does not indicate a sufficient level of detail to allow billing for a particular resource, the missing information may be supplied by SPMS 110 from the preference card data 117 or retrieved from another system by the SPMS 110. The SPMS 110 can also assist with inventory management. For example, the SPMS 110 may interface with a barcode scanner, RFID reader or similar devices and automatically record items used during a procedure which are tagged and scanned by a user prior to use or after use. The recorded items may be compared against the preference card data 117 and data in other systems such as the EHRS 120, INMS 140, ERPS 150 and/or EMBS 160 to identify inconsistencies

Various functions of the SPMS 110 such as those described above and elsewhere in this disclosure may include prompting users to take appropriate actions (e.g., creating a preference card for a scheduled procedure if none exists, reconciling inconsistencies identified during audit functions, etc.). These prompts may be generated and displayed by the user interface 114 of the SPMS 110. In some embodiments, the SPMS 110 may insert prompts or alerts in records stored by systems to which the SPMS 110 has access, such as the EHRS 120, ESS, 130 INMS 140, ERPS 150 and/or EMBS 160, or the SPMS 110 may otherwise cause those systems to provide such prompts and/or alerts to users. As an example, if information gathered by the SPMS 110 conflicts with information in the EHRS 120, the SPMS 110 may insert an alert or notation in a corresponding medical record in the EHRS 120.

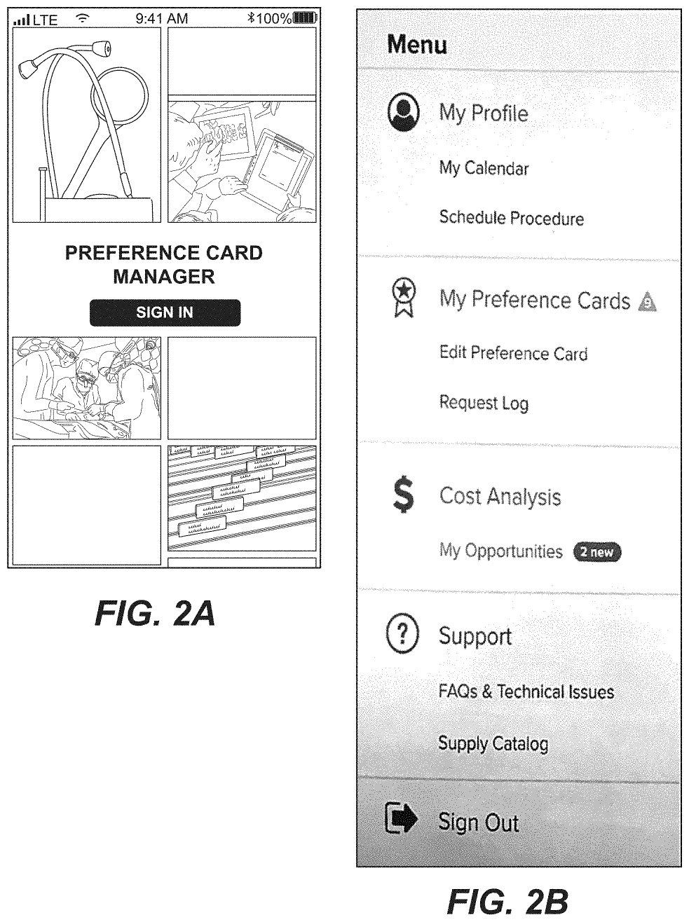

Users may manage preference cards by interacting with the SPMS 110 via the user interface 114, which will now be described in connection with example user interface displays. FIG. 2A shows an example sign-in screen allowing users to access preference cards and related information. FIG. 2B shows example user interface elements presented to a user in some such embodiments. The user interface 114 may allow the user to interact with other systems such as the EHRS 120 or ESS 130 via the SPMS 110. For example, FIG. 2B shows a selectable “My Calendar” element which allows the user to view scheduled procedures retrieved from the ESS 130. FIG. 2B also shows a selectable “Schedule Procedure” element which allows the user to schedule a new procedure via the SPMS 110, which is integrated with the ESS 130. When scheduling a procedure, the user submit the date of the procedure (and other descriptive information) to the ESS 130 via the SPMS 110. At that time, the user may also associated the scheduled event with a particular preference card retrieved from the SPMS 110 and identified by the user for use in conjunction with the procedure being scheduled. Also shown is a selectable “My Opportunities” element. As will be explained further below, the SPMS 110 may analyze cost data, resource utilization data collected and stored by the SPMS 110 as part of the preference card data 117 associated with various procedures performed by a given surgeon (who, as an example, may also be the user) and others, and present potential opportunities to edit preference cards to produce better surgical outcomes, reduced costs, or both.

Non-limiting examples of information stored in the preference card data 117 for preference card items, either as part of preference cards or part of utilization data associated with preference cards may include: descriptive names (e.g., “scalpel,” “knee implant”, et al.); manufacturer names; manufacturer part numbers; manufacturing lot number for preference card items used to refer to items within the EHRS 120, ERPS 150, EMBS 160, or other systems; clinical descriptions; unit costs; open quantities (items which should be opened and made ready for use prior to the start of a procedure); hold quantities (items which should be on hand and available for use if needed during a procedure); quantities used; locator information for the associated item(s); and so on. Non-limiting examples of information associated with procedures in the preference card data 117 may include procedure names; associated CPT or ICD-10 codes; surgeon name(s), facility name(s), procedure ID numbers, and so on.

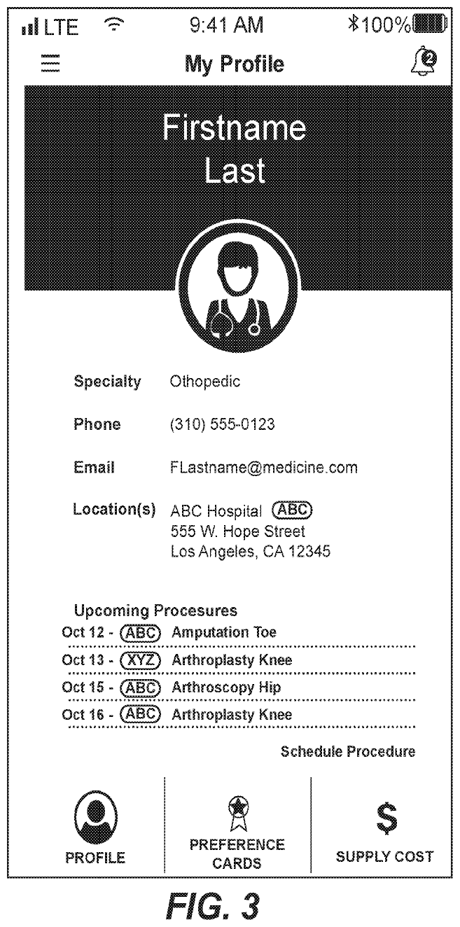

FIG. 3 shows example elements of the user interface 114 in some embodiments. The surgeon profile page allows the user to view information about the surgeon, along with a view of upcoming scheduled procedures. The user may also select elements to view a list of preference cards, schedule a procedure, or review various costs analyses.

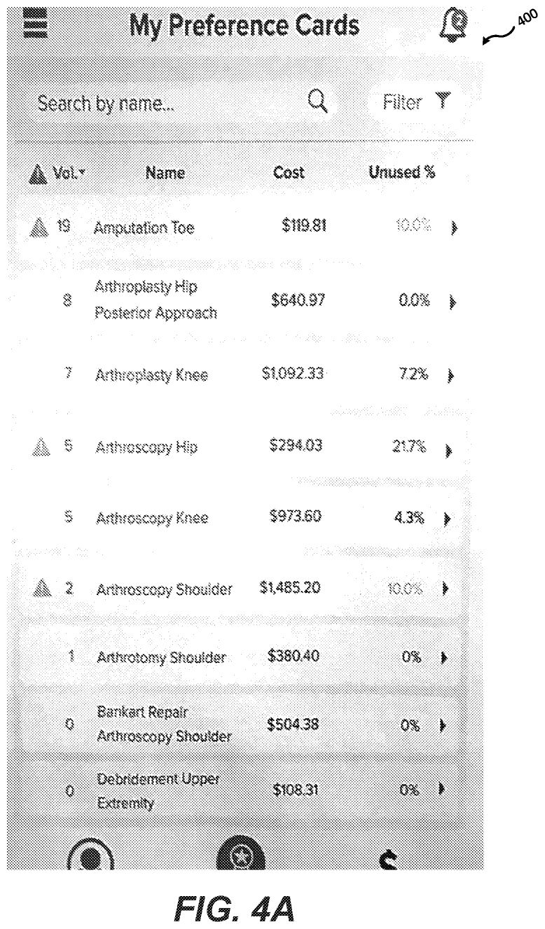

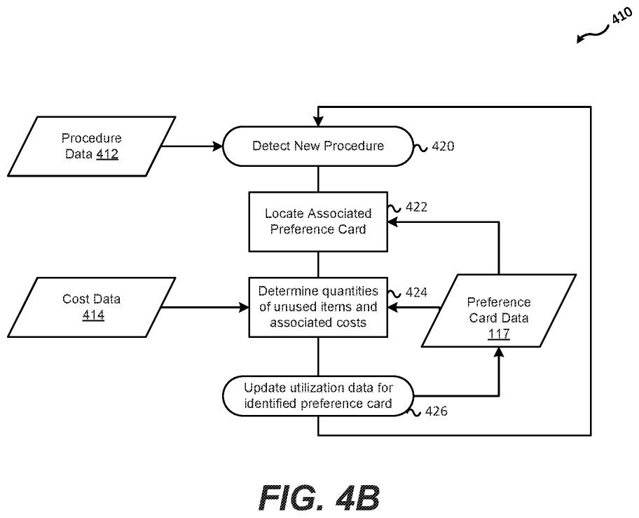

FIG. 4A shows an example user interface display 400 presented to a user, such as a surgeon, for viewing preference cards in certain embodiments. In this example embodiment, once a preference card has been generated, the SPMS 110 may augment the preference card with additional information which may be viewed by users including physicians and other staff. For example, the SPMS 110 may communicate with the ERPS 150 and/or the EMBS 160 to determine an estimated cost of the preferences expressed by the preference card, including surgical supply costs, overhead costs associated with personnel time, and overhead costs associated with the use of various facilities, such as surgical suites, as non-limiting examples.

After a medical procedure is performed, the SPMS 110 may be supplied with data from other systems, such as the EMBS 160, which can store records of procedure in association with a procedure identifier and list of item utilized for the performing of the procedure, or retrieve that data directly for analysis. In that manner, the EMBS 160 may operate a database storing historical records of completed procedures. Various analysis results can be used to measure metrics related to procedures and surgeons. For example, the SPMS 110 can interface with the ERPS 150, EMBS 160, or other systems to compare the surgical resources, such as supplies and tools that were actually used to perform procedures against the preference card used for that procedure, or against preference cards used by others for similar procedures. In the example of FIG. 4A, the SPMS 110 displays three metrics in the user interface 114 for each existing preference card: a number of previously performed procedures associated with that preference card, an estimated cost, and a percentage of unused resources. In this example, the percentage may be an aggregate metric computed over all resources specified by that preference card. As shown in FIG. 4A, the SPMS 110 may identify opportunities for improved surgical outcomes and/or lowered costs to the user via the user interface 114, as indicated by graphical “attention” icons next to certain preference cards.

In order to generate the procedure list and statistics as shown in FIG. 4A, the SPMS 110 gathers information from multiple sources in an example procedure described below. It should be understood that in various embodiments, information described as being stored by a particular system in this example may be stored in a different system instead. Thus, the description below is intended as a non-limiting example used to further illustrate details of the embodiments disclosed herein.

FIG. 4B illustrates an example procedure 410 used to produce information, such as that shown in example user interface display 400. The procedure 410 has steps 420, 422, 424, and 426. At step 420, The SPMS 110 accesses the procedure data 412. The procedure data 412 may be retrieved from one or more sources such as the EHRS 120 and/or ESS 130 (and/or other systems) and is used to determine that a procedure has been performed corresponding to a particular preference card.

At step 422, the SPMS 110 retrieves the preference card used for the procedure from the preference card data 418 managed by the SPMS 110. At step 424, the SPMS 110 retrieves the cost data 414 for the preference card items and the items actually used during the procedure. The cost data 414 may be based upon one or more data sources. For example, the cost data may be based entirely upon records stored by the ERPS 150, or, in another example, the cost data may be synthesized from procurement data stored by the REPS containing records of the items procured for the procedure and billing data stored by the EMBS 160 containing records of items whose use during the procedure was billed. The SPMS 110 uses the preference card data 117, as well as the cost data 414, to determine costs incurred for any items specified by the preference card, but unused during the procedure, by comparing actual items and quantities used (as determined from the EPRS and/or the EMBS 160) and compares them with the items and quantities requested by the preference card. The SPMS 110 may also retrieve records from the EMBS 160 indicating which items were billed. Discrepancies may exist when not all procured items are used during the procedure. In some instances, items made ready for use during the procedure cannot be reused, even if they are not used in the procedure. In other instances, there may be costs associated with preparing an unused item for re-use (e.g., sterilizing and repackaging a reusable surgical tool). In addition, the SPMS 110 may detect instances where additional resources are procured for a procedure which were not requested in the preference card for that surgery and add the costs of those items.

At step 424, the SPMS 110 updates the preference card data 117 to include the results of the procedure. If the preference card data 117 already stores information for preference cards associated with the same procedure type as the current procedure, the various metrics are updated to reflect the impact of the current procedure. The metrics may include, as non-limiting examples, the cost of the items utilized, cost of unused items, percentage of items requested but not utilized, costs relative to average costs for previous uses of the current preference cards, costs relative to average costs for uses of similar preference cards associated with other users.

The SPMS 110 maintains and updates aggregate statistics for each procedure it detects, enabling the SPMS 110 to display the augmented preference card data, such as the data shown in FIG. 4A, which includes the number of procedures performed by a user, the average (or expected) costs of each procedure, and an average percentage of wasted or unused resources associated with each procedure (when performed by a particular user and associated with a particular preference card). Procedures may be identified by associated CPT, ICD, or other coding stored in by the EHRS 120 and EMBS 160.

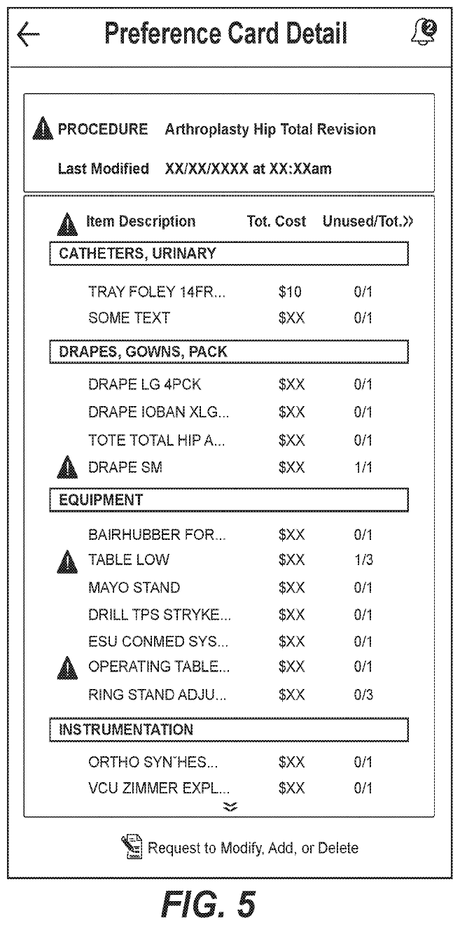

In various embodiments, the user may select a particular preference card in the user interface 114, as shown in FIG. 4A, to display a detailed breakdown for the selected preference card, as shown by the example display in FIG. 5.

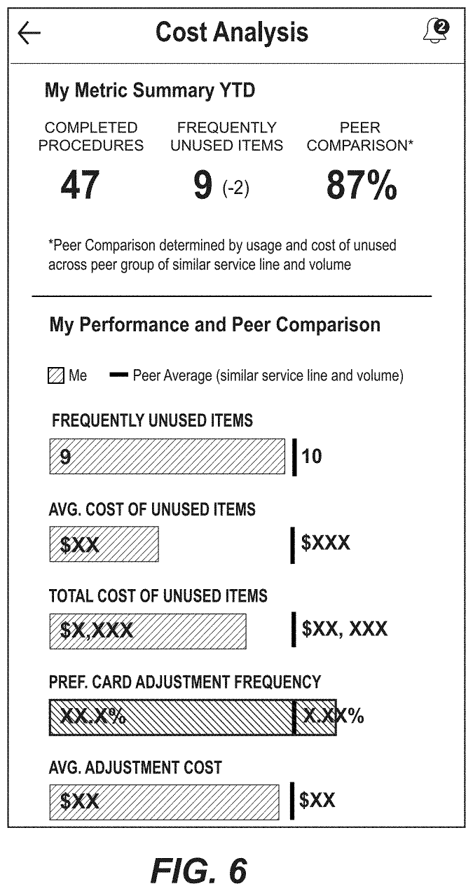

FIG. 6 is an example display of metrics calculated by the SPMS 110. For example, FIG. 6 shows the number of surgical resources which are frequently included in a surgeon's preference card(s) but not used during associated procedures. This and other metrics may be presented along with an indicator of how each metric value compares to a surgeon's peers. Other information shown in the example display of FIG. 6 includes a surgeon's total number of complete procedures, the change in the surgeon's number of frequently unused items over a given time period, and a measure of their performance compared to their peers. Also shown are average costs for unused items, aggregate costs of unused items, frequency with which supplemental items are required during procedures and the average cost impact of those supplemental items. It should be understood that the elements shown in FIG. 6 and elsewhere are non-limiting examples of useful metrics which can be identified and tailored to specific scenarios and institutions. For example, a particular facility may wish to reduce the occurrence of postoperative complications. In this example, the SPMS 110 may be configured to quantify the occurrence of complications and display related metrics to users.

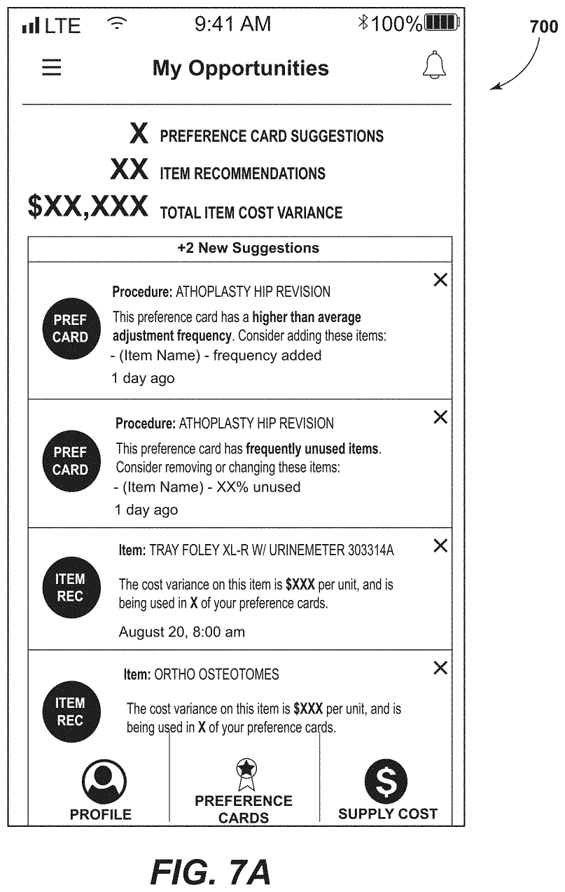

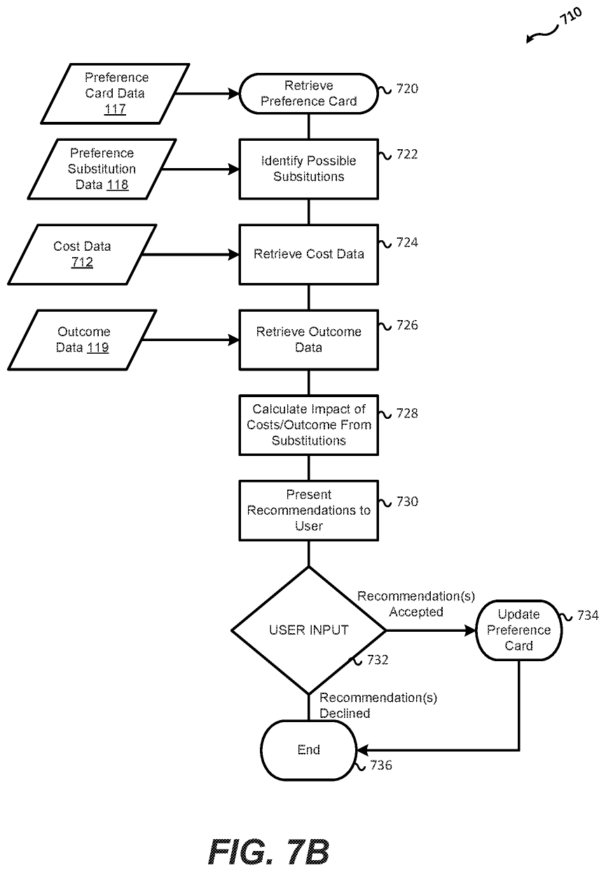

FIG. 7A illustrates an example user interface display 700 identifying opportunities for a user to modify preference cards based on potential opportunities for improvement identified by the SPMS 110 using customizable rules and metrics. As shown in the example user interface display 700, the SPMS 110 indicates to the user via the user interface 114 that the surgical resources specified by the “ATHROPLASTY HIP REVISION” preference card are frequently subject to change during performance of the associated procedure. That is, the system may determine that the percentage of times a particular procedure is performed and the resources utilized in performing the procedure differ from those listed in the associated preference exceeds a particular pre-determine threshold. For instance, the preference card may not specify an adequate quantity of one or more supplies or tools. In this example, time (and hence costs) might be saved if the preference card were updated to make the need to retrieve additional supplies during the procedure less frequent. As further shown in FIG. 7A, the SPMS 110, may also indicate that a particular item has a high cost (or high variance). The example suggestions may be selectable within the user interface 114 allowing a user to accept suggestions (and modify them if desired) within the user interface 114, causing the appropriate preference cards to be updated by the SPMS 110. Alternative items may be identified by the SPMS 110 (e.g., specifically, the enterprise resource planning system 150) storing listing of substitutable equipment items. When a user adds a particular items to a preference card or simply views the contents of a preference card, therefore, the SPMS 110, via user interface 114 can analyze the listing of items in the preference, determine a set of suitable alternative items (e.g., as identified in enterprise resource planning system 150), and determine whether those items may be more cost effective. If so, the alternative items could be displayed to the user as potential substitutes to add to a particular preference card or to replace a particular item in a preference card.

In some embodiments wherein the SPMS 110 recommends alternatives to various preference card items, the SPMS 110 accesses a preference substitution data 118 indicating preference card items which have alternatives and one or more alternative preference items (e.g., a scalpel blade ‘A’ made by manufacturer ‘X’ and a scalpel blade ‘B’ made by manufacturer ‘Y’). In some cases, this data may be specific to a type of procedure. For example, scalpel blade ‘B’ may only be a suitable substitute for scalpel blade ‘A for certain surgeries. In some embodiments, the substitute preference data may be even more specific, including factors corresponding to patient or condition information accessed from the EHRS 120. For instance, the use of a particular item, such as anesthetic ‘B’, may be contraindicated for certain patient demographics, or patients with certain conditions. In this instance, the database entry for anesthetic ‘A’ would indicate that anesthetic ‘B’ is a suitable substitute unless the preference card is associated with a procedure to be performed on a patient with a contraindicated condition. The SPMS 110 may access the ESS 130 and EHRS 120 to determine whether the patient has the contraindicated condition, based on searching the patient's record for a corresponding diagnosis code, or automated textual analysis. If an appropriate substitute for a particular item on a preference card is available, the SPMS 110 may calculate the cost difference if the substitution is made and recommend the substitution if the difference would result in savings greater than a predetermined threshold input by a user.

In certain embodiments, the SPMS 110 may present different levels of information via the user interface 114 depending on the location or device in which the user interface 114 is presented. For example, the SPMS 110 may prevent the display of any sensitive patient health information if the device or location from which the SPMS 110 being accessed is insecure. The location of the device displaying user interface 114 may be established using any suitable device-locating technology, such as global positioning system (GPS) technology or Internet protocol (IP) address-based device locating technology. In such an embodiment, the SPMS 110 may define particular geographical zones in which the display of sensitive patient health information is authorized (e.g., one such zone may overlay a particular hospital location). In that case, the SPMS 110 determines the location of the device accessing the SPMS 110. If that location falls within an authorized zone, the display of sensitive information is authorized and the SPMS 110 will transmit such information to the user's device. But if the device location falls outside the defined authorized zone, the SPMS 110 may inhibit the transmission of such information to the device. In other embodiments, the SPMS 110 may ensure that sensitive patient health data is never displayed via the user interface 114.

As discussed above, the SPMS 110 may be provided with additional information, such as the outcome data 119. The outcome data 119 may identify surgical preferences which influence surgical outcomes. For example, one surgeon may prefer to use stitches for a laceration and another may prefer surgical glue. The SPMS 110 may access general data about outcomes of laceration repairs using different approaches and determine that a surgeon should consider switching from stiches to surgical glue. The recommendations may be tailored to particular circumstances. For instance, the SPMS 110 may access ICD and/or CPT coding data (and/or other information) from the EHRS 120. As a particular example, switching from stiches to surgical glue may not be indicated for all laceration injuries, or for certain patients with additional injuries or underlying conditions. In this example, the SPMS 110 may assess whether the preference card for a laceration repair should be modified for all laceration repairs performed by a particular surgeon, or only on a case-by-case basis.

In embodiments where the SPMS 110 makes recommendations based on outcomes, the SPMS 110 may access a surgical outcomes database having entries indicating whether a particular preference card item is known (or believed to be correlated) with one or more outcome metrics and whether that correlation is positive or negative. When a substitute preference card item is available for a given preference card item, the SPMS 110 may determine whether the substitution is likely to result in an improved outcome and, if so, recommend the substitution. The outcome data 119 may be supplied to the SPMS 110 and/or the SPMS 110 may actively generate the outcome data 119. For example, the SPMS 110 may repeatedly analyze historical and current data retrieved from the EHRS 120 indicating procedure outcomes and identify trends using various computational analysis techniques, including natural language processing, sentiment analysis, and advanced machine learning algorithms. Non-limiting examples of information stored in the outcomes data 119 may include morbidity and mortality rates, recovery times, and patient satisfaction scores.

In some embodiments, the SPMS 110 optionally provides data relating to a recommended substitution to the user along with the recommendation in addition to cost data. As an example, the system may recommend bone cement ‘B’ as a replacement for bone cement ‘A’ along with outcome data indicating that surgical outcomes are equivalent between procedures using bone cement ‘A’ and those using bone cement ‘B.’ The data may be retrieved from the outcome data 119 or from an external database or search engine.

FIG. 7B illustrates an example procedure 710 performed by the SPMS 110 to make recommendations as illustrated in FIG. 7A and described above. The procedure 710 includes the steps 720, 722, 724, 726, 728, 730, 734, and 736. At step 720, the SPMS 110 retrieves preference card data 117 corresponding to a preference card selected by a user. Alternatively, the steps 720-728 may also be performed continuously for numerous preference cards so recommendations can be provided instantaneously when a user accesses the SPMS 110.

At step 722, the SPMS 110 retrieves the preference substitution data 118 for each item listed in the preference card data 117 retrieved at step 720. The SPMS 110 uses the preference substitution data 118 to identify possible substitutions subject to constraints specified in advance, such as those described above.

At step 724, the SPMS 110 retrieves the cost data 712 from the ERPS 150 and/or the EMBS 160 for the original preference card items as well as the possible substitute items. Similarly, at step 726, the SPMS 110 retrieves the outcome data 119 from an outcomes database (described above in connection with FIG. 7A), for the original preference card items as well as the possible substitute items.

At step 728, the SPMS 110 computes the expected impact of the various substitutions. In the case of cost impacts, the SPMS 110 may display expected decreases (or increases) in cost in currency amounts or as percentage decreases (or increases). Outcome impacts may be reported in any number of ways, non-limiting examples of which include decreases (or increases) in expected recovery time, pain scores, likelihood of complications, and so on.

At step 730, the impacts are presented to the user via the user interface 114, as illustrated by the example user interface display 700 of FIG. 7A. At step 732, the user interface 114 allows the user to accept or decline various suggestions. If the user declines all suggestions, the procedure concludes at step 736. If the user accepts one or more suggestions, the procedure continues to step 734 and updates the preference card before concluding at step 736.

In embodiments, a system includes a preference card data store storing a plurality of preference card records. Each preference card record in the plurality of preference card records is associated with at least one disease identifier and at least one procedure identifier. The system includes a computer server including processing circuitry and a memory. The memory stores instructions that, when executed by the processing circuitry, cause the processing circuitry to perform the steps of receiving, using a communications network, a first data request from a remote computer system, the first data request encoding a procedure identifier, executing a database lookup operation in the preference card data store using the procedure identifier to identify a first set of preference cards, wherein each preference card in the first set of preference cards is associated with a procedure identifier that matches the procedure identifier encoded into the first data request, encoding a user interface including descriptions of each of the preference cards in the first set of preference cards, and transmitting, via the communications network, the user interface to the remote computer system.

The system may include an electronic scheduling system in which the memory stores instructions that, when executed by the processing circuitry, cause the processing circuitry to perform the steps of receiving, via the communications network and from the remote computers system, a second data request identifying a first preference card of the first set of preference cards and an identification of a scheduled procedure, and executing a database operation in the electronic scheduling system to create a database record associating the scheduled procedure with the first preference card. The memory may further store instructions that, when executed by the processing circuitry, cause the processing circuitry to perform the steps of determining, for a first preference card in first set of preference cards, a minimum set of data elements based on the procedure identifier associated with the first preference card, retrieving, from the preference card data store, a set of data elements associated with the first preference card, determining a first data element that is included in the minimum set of data elements that is not included in the set of data elements associated with the first preference card, and encoding into the user interface an identification of the first data element. The memory may further store instructions that, when executed by the processing circuitry, cause the processing circuitry to perform the steps of receiving, from the remote computer system, an electronic message encoding a value for the first data element, and executing a database operation in the preference card data store to create a database record associating the value with the first preference card.

The system may include a historical procedure database storing a plurality of completed procedure records, wherein each completed procedure record identifies a set of items utilized in completion of the procedure and an associated procedure identifier and a first preference card in the first set of preference cards identifies a set of items to be used in performing the procedure associated with the first preference card. The memory may store instructions that, when executed by the processing circuitry, cause the processing circuitry to perform the step of, for a first item in the set of items identified by the first preference card, accessing the historical procedure database to determine a percentage of completed procedure records that are associated with a procedure identifier that matches the procedure identifier associated with the first preference card and identify the first item in the set of items utilized in completion of the procedure. The memory may store instructions that, when executed by the processing circuitry, cause the processing circuitry to perform the step of encoding an alert into the user interface when the percentage falls below a predetermined threshold. In embodiment, the first preference card in the first set of preference cards includes at least one of a common procedural technology code and an international classification of disease code.

In some embodiments, the memory stores instructions that, when executed by the processing circuitry, cause the processing circuitry to perform the steps of determining a location of the remote computer system, and, when the location of the remote computer system falls outside a predetermined geographic region, preventing the processing circuitry from incorporating patient data into the user interface. The memory may store instructions that, when executed by the processing circuitry, cause the processing circuitry to perform the steps of determining a first item identified by a first preference card in the first set of preference cards, accessing a substitution database to identify a first substitute item for the first item identified by the first preference card, and encoding an identification of the first substitute item into the user interface.

In an embodiment, a method includes receiving, using a communications network, a first data request from a remote computer system, the first data request encoding a procedure identifier, executing a database lookup operation in a preference card data store using the procedure identifier to identify a first set of preference cards, wherein each preference card in the first set of preference cards is associated with a procedure identifier that matches the procedure identifier encoded into the first data request, encoding a user interface including descriptions of each of the preference cards in the first set of preference cards, and transmitting, via the communications network, the user interface to the remote computer system.

In an embodiment, a method includes a method of managing surgical preferences, comprising receiving preference cards via processing circuitry of a surgical preference management system (SPMS). Each preference card includes a set of surgical preferences associated with a particular procedure type and a particular surgeon. The surgical preferences specify quantities of surgical resources desired by the surgeon when performing the particular procedure type. The method includes determining a cost for each surgical resource in each preference card by identifying a procurement record corresponding to that surgical resource in an enterprise resource planning system (ERPS) based on a resource description of that surgical resource in that preference card, and retrieving an estimated cost from the identified record, retrieving a record indicating that a particular surgical resource may be substituted by an alternative surgical resource from substitute preference data stored by the SPMS, and displaying a recommendation via a user interface supplied by the SPMS to alter a preference card selected by a user of the SPMS, based on one of the following: determining, using a record retrieved from the ERPS, that a cost of the alternative surgical resource is lower than a cost of the particular surgical resource, determining, using a record retrieved from an outcomes database stored by the SPMS, that a procedure outcome metric of the alternative surgical resource is better than an procedure outcome metric of the particular surgical resource, determining, using utilization data collected by the SPMS and associated with the selected preference card, that average costs of one or more resources consumed during surgical procedures associated with the selected preference card is greater than numbers of the one or more specified in the selected preference card by more than a first predetermined threshold, and determining, using utilization data collected by the SPMS and associated with the one or more other preference cards, that average costs of surgical procedures associated with the selected preference card are greater than average costs of surgical procedures associated with the one or more other preference cards. The method includes receiving an instruction from the user via the user interface to implement one or more of the recommended alterations to the particular preference card and implementing the one or more recommended alterations by updating the selected preference card, and causing the updated selected preference card to be used to automatically procure surgical resources for a surgical procedure associated with the selected preference card.

A reader skilled in the relevant art will appreciate that numerous advantages may be realized by practicing methods and utilizing systems disclosed herein. For example, digitally storing, editing, transmitting, and implementing surgical preference cards is expected to produce significant time and cost savings due to increased automation alone. Furthermore, improved patient outcomes, cost savings, and improvement in any number of other metrics may be realized by consolidating preference card data, obtaining relevant information from related medical and business systems, and performing static and temporal statistical analyses to identify opportunities which might otherwise go unnoticed. Presenting relevant information and actionable suggestions directly to users through a user interface allows users to immediately act upon those suggestions and increases the likelihood that opportunities for improvement are realized both initially and on an ongoing basis.

The invention claimed is:

1. A system, comprising:
a preference card data store storing a plurality of preference card records, each preference card record in the plurality of preference card records being associated with at least one disease identifier and at least one procedure identifier; and
a computer server, including:
processing circuitry, and
a memory storing instructions that, when executed by the processing circuitry, cause the processing circuitry to perform the steps of:
receiving, using a communications network, a first data request from a remote computer system, the first data request encoding a procedure identifier,
executing a database lookup operation in the preference card data store using the procedure identifier to identify a first set of preference cards, wherein each preference card in the first set of preference cards is associated with a preference card record including a procedure identifier that matches the procedure identifier encoded into the first data request,
encoding a user interface including descriptions of each of the preference cards in the first set of preference cards, and
transmitting, via the communications network, the user interface to the remote computer system.

2. The system of claim 1, further comprising an electronic scheduling system and wherein the memory stores instructions that, when executed by the processing circuitry, cause the processing circuitry to perform the steps of:
receiving, via the communications network and from the remote computers system, a second data request identifying a first preference card of the first set of preference cards and an identification of a scheduled procedure; and
executing a database operation in the electronic scheduling system to create a database record associating the scheduled procedure with the first preference card.

3. The system of claim 1, wherein the memory stores instructions that, when executed by the processing circuitry, cause the processing circuitry to perform the steps of:
determining, for a first preference card in first set of preference cards, a minimum set of data elements based on the procedure identifier associated with the first preference card;
retrieving, from the preference card data store, a set of data elements associated with the first preference card;
determining a first data element that is included in the minimum set of data elements that is not included in the set of data elements associated with the first preference card; and
encoding into the user interface an identification of the first data element.

4. The system of claim 3, wherein the memory stores instructions that, when executed by the processing circuitry, cause the processing circuitry to perform the steps of:
receiving, from the remote computer system, an electronic message encoding a value for the first data element; and
executing a database operation in the preference card data store to create a database record associating the value with the first preference card.

5. The system of claim 1, further comprising a historical procedure database storing a plurality of completed procedure records, wherein each completed procedure record identifies a set of items utilized in completion of the procedure and an associated procedure identifier and a first preference card in the first set of preference cards identifies a set of items to be used in performing the procedure associated with the first preference card.

6. The system of claim 5, wherein the memory stores instructions that, when executed by the processing circuitry, cause the processing circuitry to perform the step of, for a first item in the set of items identified by the first preference card, accessing the historical procedure database to determine a percentage of completed procedure records that are associated with a procedure identifier that matches the procedure identifier associated with the first preference card and identify the first item in the set of items utilized in completion of the procedure.

7. The system of claim 6, wherein the memory stores instructions that, when executed by the processing circuitry, cause the processing circuitry to perform the step of encoding an alert into the user interface when the percentage falls below a predetermined threshold.

8. The system of claim 5, wherein the first preference card in the first set of preference cards includes at least one of a common procedural technology code and an international classification of disease code.

9. The system of claim 1, wherein the memory stores instructions that, when executed by the processing circuitry, cause the processing circuitry to perform the steps of:
determining a location of the remote computer system; and
when the location of the remote computer system falls outside a predetermined geographic region, preventing the processing circuitry from incorporating patient data into the user interface.

10. The system of claim 1, wherein the memory stores instructions that, when executed by the processing circuitry, cause the processing circuitry to perform the steps of:
determining a first item identified by a first preference card in the first set of preference cards;
accessing a substitution database to identify a first substitute item for the first item identified by the first preference card; and
encoding an identification of the first substitute item into the user interface.

11. A method, comprising:
receiving, using a communications network, a first data request from a remote computer system, the first data request encoding a procedure identifier;
executing a database lookup operation in a preference card data store using the procedure identifier to identify a first set of preference cards, wherein each preference card in the first set of preference cards is associated with a procedure identifier that matches the procedure identifier encoded into the first data request;
encoding a user interface including descriptions of each of the preference cards in the first set of preference cards; and
transmitting, via the communications network, the user interface to the remote computer system.

12. The method of claim 11, further comprising:
receiving, via the communications network and from the remote computers system, a second data request identifying a first preference card of the first set of preference cards and an identification of a scheduled procedure; and
executing a database operation in an electronic scheduling system to create a database record associating the scheduled procedure with the first preference card.

13. The method of claim 11, further comprising:
determining, for a first preference card in first set of preference cards, a minimum set of data elements based on the procedure identifier associated with the first preference card;
retrieving, from the preference card data store, a set of data elements associated with the first preference card;
determining a first data element that is included in the minimum set of data elements that is not included in the set of data elements associated with the first preference card; and
encoding into the user interface an identification of the first data element.

14. The method of claim 13, further comprising:
receiving, from the remote computer system, an electronic message encoding a value for the first data element; and
executing a database operation in the preference card data store to create a database record associating the value with the first preference card.

15. The method of claim 11, further comprising, for a first item in a set of items identified by the first preference card, accessing a historical procedure database to determine a percentage of completed procedure records that are associated with a procedure identifier that matches the procedure identifier associated with the first preference card and identify the first item in the set of items utilized in completion of the procedure.

16. The method of claim 15, further comprising encoding an alert into the user interface when the percentage falls below a predetermined threshold.

17. The method of claim 11, wherein the first preference card in the first set of preference cards includes at least one of a common procedural technology code and an international classification of disease code.

18. A method of managing surgical preferences, comprising:
receiving preference cards via processing circuitry of a surgical preference management system (SPMS), each preference card including a set of surgical preferences associated with a particular procedure type and a particular surgeon, the surgical preferences specifying quantities of surgical resources desired by the surgeon when performing the particular procedure type;
determining a cost for each surgical resource in each preference card by identifying a procurement record corresponding to that surgical resource in an enterprise resource planning system (ERPS) based on a resource description of that surgical resource in that preference card, and retrieving an estimated cost from the identified record;
retrieving a record indicating that a particular surgical resource may be substituted by an alternative surgical resource from substitute preference data stored by the SPMS;
displaying a recommendation via a user interface supplied by the SPMS to alter a preference card selected by a user of the SPMS, based on one of the following:
determining, using a record retrieved from the ERPS, that a cost of the alternative surgical resource is lower than a cost of the particular surgical resource;
determining, using a record retrieved from an outcomes database stored by the SPMS, that a procedure outcome metric of the alternative surgical resource is better than an procedure outcome metric of the particular surgical resource;
determining, using utilization data collected by the SPMS and associated with the selected preference card, that average costs of one or more resources consumed during surgical procedures associated with the selected preference card is greater than numbers of the one or more specified in the selected preference card by more than a first predetermined threshold; and
determining, using utilization data collected by the SPMS and associated with the one or more other preference cards, that average costs of surgical procedures associated with the selected preference card are greater than average costs of surgical procedures associated with the one or more other preference cards;
receiving an instruction from the user via the user interface to implement one or more of the recommended alterations to the particular preference card and implementing the one or more recommended alterations by updating the selected preference card; and
causing the updated selected preference card to be used to automatically procure surgical resources for a surgical procedure associated with the selected preference card.

19. The method of claim 18, wherein the preference cards include at least one of a common procedural technology code and an international classification of disease code.

20. The method of claim 18, further comprising:
identifying a scheduled procedure in an electronic scheduling system; and
associating the updated selected preference card with the scheduled procedure.
